(19) United States
(12) Patent Application Publication
(10) Pub. No.: US 2023/0142905 A1
(43) Pub. Date: May 11, 2023

(54) METHOD FOR PREDICTING THE RETURN TO FUNCTIONAL AUTONOMY IN A SUBJECT SUFFERING FROM AN ACUTE EVENT

(71) Applicants: SORBONNE UNIVERSITE, Paris (FR); APHP (ASSISTANCE PUBLIQUE - HÔPITAUX DE PARIS), Paris (FR); INSERM (INSTITUT NATIONAL DE LA SANTÉ ET DE LA RECHERCHE MÉDICALE), Paris Cedex 13 (FR)

(72) Inventors: Delphine SAUCE, Saint-Maur-des-Fossés (FR); Martin LARSEN, Saint-Maur-des-Fossés (FR); Jacques BODDAERT, Paris (FR); Hélène VALLET, Villemomble (FR)

(73) Assignees: SORBONNE UNIVERSITE, Paris (FR); APHP (ASSISTANCE PUBLIQUE - HÔPITAUX DE PARIS), Paris (FR); INSERM (INSTITUT NATIONAL DE LA SANTÉ ET DE LA RECHERCHE MÉDICALE), Paris Cedex 13 (FR)

(21) Appl. No.: 17/918,686

(22) PCT Filed: Apr. 23, 2021
(86) PCT No.: PCT/EP2021/060724

§ 371 Date: Oct. 13, 2022

(30) Foreign Application Priority Data
Apr. 24, 2020 (EP) 20305403.6

(51) Int. Cl. G01N 33/53

(52) U.S. Cl. CPC G01N 33/5308; G01N 2800/52

(57) ABSTRACT

A method for predicting the return to functional autonomy in a subject suffering from an acute event, including the steps of i) determining neopterin level in a biological sample obtained from the subject; ii) comparing the level with its predetermined reference neopterin level and iii) predicting that the subject will have a long time to return to functional autonomy when the level of neopterin is higher than its predetermined reference neopterin level or predicting that the subject will have a short time to return to functional autonomy when the level of neopterin is lower than its predetermined reference neopterin level.

The present application is in the field of geriatric medicine. The application pertains generally to methods of monitoring of a patient, and more particularly to a method for predicting the length of time required for the return to functional autonomy of a subject, preferably an elderly subject, following an acute event such as a fracture.

The WHO estimates 88% increase in the number of people older than 65 over the next 20 years due to an aging world population, better public health and medical interventions that extend life expectancy. Frail older adults are at high risk of major adverse health outcomes, including disability, falls, institutionalization, hospitalization, and mortality. One of the complications of frail elderly is hip-fracture (HF). Thus, the projected rise of 15% in hip fracture incidence by 2030, combined with frailer elderly and socially dependent patients who are likely to experience greater rates of morbi-mortality and longer inpatient stays, has implications for healthcare strategy and resource allocation. Nowadays, consequences of HF are devastating with 50% of patients becoming dependent and 30% dying within one-year post surgery.

Indeed, HF patients belong to a vulnerable group of old people with comorbid diseases and a high risk of postoperative morbidity and mortality. The number of annual HF worldwide is actually 1.6 million, but is expected to reach more than 4 million in 2050, and has an estimated annual cost >$8 billion/year for inpatients only. This may reach S 14 billion/year in US if including the costs that can be incurred by rehabilitation/rehospitalization over a period of one year after HF. Consequently, a major challenge is the management of the health and socio-economic burden caused by this acute physical stress in the older population (+75 years). According to Bouchon's definition, HF represents an acute event that can participate to the accelerated decline of health in the elderly.

Today, clinical decision-making post-HF surgery is mainly based on medical parameters since no biomarkers can be used to decide on adapted care for HF patients. Many scores have been proposed but they have not been clinically useful without external validation. As such, clinician's intuition on HF patients illustrates the parable of the “blind men and an elephant”. A careful preoperative assessment is usually recognized as essential in these patients to stabilize coexisting medical conditions, though delaying surgery is associated with a poorer outcome. Lastly, there is no alternative to surgery that must be finally done. Thus, most organizational and medical reasons may not be appropriate taking into account the urgent need for surgery.

Thus, there is a need in the art for simple, efficient and reliable methods for classifying patients according to their ability to recover their functional autonomy.

Inventors have previously shown that pre-operative levels of the biomarker neopterin pinpoints high-risk patients since its plasmatic concentration is predictive of early mortality.

In the present application, the inventors show that pre-operative neopterin also pinpoints high-risk patients since its concentration is predictive of the capacity of a patient to recover from a loss of autonomy following an acute event. No correlation was previously made between neopterin concentration and return to functional autonomy.

A first object of the present invention is hence a method for predicting the return to functional autonomy in a subject suffering from an acute event. Such a method can be used in the emergency ward and/or during the hospital stay and/or after the discharge from the hospital.

A second object of the invention is a method of treatment, comprising the steps of the method for predicting the return to functional autonomy according to the first aspect and comprising an additional step of adapting the treatment.

Another object of the invention is a kit for predicting the return to functional autonomy in a subject suffering from an acute event.

Measuring the level of neopterin in emergency ward should enable stratification and improvement of patient selective triage by directing high risk patients to a specific orthogeriatric unit that has been shown to be a cost-effective adapted health solution.

Previously, physicians most likely directed patients toward these units on a first come, first served basis, or with the help of inaccurate markers and/or their own expertise. As a consequence, patients which would benefit the most from these units are not preferentially directed to these.

According to another aspect of the invention, measuring the level of neopterin after the discharge of the patient should enable practitioner to assess and adapt the post-surgery program to improve the recovery from the event and the surgery, thus improving the overall health of the patient and hastening its return to functional autonomy.

### Definitions

“Acute event” refers to any accidents, burn or traumas from which a subject, typically an elderly suffers. The term “accident” includes any unforeseen and unplanned event or circumstance resulting in damage inflicted on the body by an external force including resulting from fall or physical violence. The term “trauma” as used herein encompasses a damage which interrupts the integrity of the body skin or bone like a wound, particularly a wound that may be associated with tearing, cutting, piercing, or breaking of the tissue. In a particular embodiment, the acute event is selected from the group consisting of: hip fracture, pelvic fracture, tibia fracture, fibula fracture, foot fracture, wrist fracture, clavicle fracture and hand fracture.

In the context of the invention, an acute event does not comprise diseases or complications associated with a preexisting condition.

In the context of the invention, an acute event has a unique, external, identifiable source (for example a fall, a fire, a kitchen instrument), rather than an underlying condition or a combination of circumstances leading to a complication.

“Functional autonomy” refers to a state where the subject is free from any complication which render the subject unable to perform most of the basic movements and/or acts necessary to everyday life, and therefore free of any need for care from a third party.

Functional autonomy is unrelated to overall health (i.e. lack of illness and/or disability).

To determine whether a subject is functionally autonomous (i.e. in a state of functional autonomy), an array of generally accepted tests and indexes are available to the person skilled in the art, and are usable independently or in conjunction with one another.

These tests and indexes offer a subjective way of defining if a subject is functionally autonomous or not, by measuring without bias and without relying on the testimony of the subject or a third party the subject's capacities.

A list of such tests and indexes comprises but is not limited to SPPB (Short Physical Performance Battery) (Guralnik et al. 1994), Barthel's index (Barthel et al. 1964), “Get up and go” test (Podsiadlo et al. 1991), ADL (Baltes et al., 1996; Baltes et al., 1993), IADL (Baltes et al., 1993, 1996) Katz′ Index (Katz S, Down TD, Cash HR. Progress in the development of the index of ADL. Gerontologist 1970; 10:20-30), Lawton's test (Lawton M, Brody EM. Assessment of older people: self-maintaining and instrumental activities of daily living. Gerontologist 1969; 9:179-86.), or AGGIR's grid (Syndicat National de Gérontologie Clinique. AGGIR. Guide pratique pour 1a codification des variables. Principaux profils des groupes isoressources. Revue Geriatr 1994; 19:249-59.)

For the purpose of example, such movements and/or acts comprises but are not limited to:
Getting up from a lying, a reclining or a sitting position,
Performing the basic hygiene acts,
Cooking or feeding itself,
Putting on clothes,
Completing the basic cleaning tasks.

In this regard, determining the functional autonomy of a patient is a well-known exam of the person skilled in the art.

“Return to functional autonomy” refers to the duration spanning from the acute event to the state of functional autonomy as defined above.

The return to functional autonomy being based on the healing process of both the fracture and the surgery, the person skilled in the art is capable, based on the teaching of the present invention, to apply the protocol hereunder described to any acute event.

In the context of the invention, the subject suffers from a hip fracture. The skilled person in the art can determine, based on the information provided by the method herein described, whether the treatment of subject suffering from hip fracture must be adapted, in particular if the subject has to be directed to a specialized care unit. Such a specialized care unit is, in a non-limiting fashion, a care unit where patients are under the supervision of both a physician specialized in geriatric and one specialized in orthopedics, or under the supervision of one of these physicians with regular examination from the other specialty. UPOG (“Unité péri-opératoire gériatrique”—geriatric perioperative unit) in France is an example of these specialized care units.

“Neopterin” refers to 2-amino-4-hydroxy-6-(D-erythro-, 2′, 3′-trihydroxypropyl) which has the following Formula I and belongs to the group of pteridines. Neopterin represents a precursor molecule of biopterin that is an essential cofactor in neurotransmitter synthesis, and it is also involved in a variety of oxydation/reduction reactions in the body. Neopterin is derived in vivo from guanosine triphosphate (GTP). The enzyme GTP-cyclohydrolase-I (GTPCH I) catalyses this reaction in activated monocytes, macrophages, dendritic cells, and endothelial cells and to a lesser extent in renal epithelial cells, fibroblasts, and vascular smooth muscle cells upon stimulation mainly by interferon gamma and to a lesser extent by interferon alpha and beta with its release being enhanced by tumor necrosis factor.

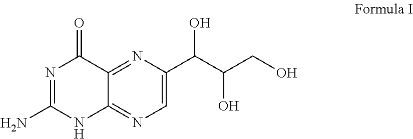 [SMILES: Nc1nc(=O)c2nc(C(O)C(O)CO)cnc2[nH]1]

A “sample” refers to sample obtained from a subject, for example blood, saliva, feces, urine, semen, blood plasma, synovial fluid or serum. In a particular embodiment, the biological sample is blood sample. The term “blood sample” means any blood sample derived from the subject. Typically, the level of neopterin is measured in a blood sample obtained when the subject arrived at hospital. In another embodiment, the blood sample is cryo-preserved at −80° C. and the level of neopterin may be measured up to 36 months starting from the cryo-preservation. Typically, the serum has been obtained after collecting blood by venipuncture, allowing clotting. The clot is removed by centrifugation at room temperature and the resulting supernatant, designated serum, is carefully removed using Pasteur pipette. Plasma is produced when whole blood is collected into tubes that are treated with anticoagulant (heparine).

A “fresh frozen” sample refers to a sample which has been frozen less than 48 hours after its collection.

A “subject” is a human, either male or female. In a particular embodiment, the subject is an elderly. As used herein, the term “elderly” refers to a subject more than 65 years-old, more preferably more than 75 years-old.

In particular embodiment, the subject suffers or is susceptible to suffer from a hip fracture, pelvic fracture, tibia fracture, fibula fracture, foot fracture, wrist fracture, clavicle fracture and/or hand fracture.

“Enzyme-linked immunosorbent assays” (ELISA), refers to an assay using a solid-phase enzyme immunoassay to detect the presence of a ligand (commonly a protein) in a liquid sample using antibodies directed against the protein to be measured.

ELISA, in general, are performed by binding a reference reagent (antigen) to a solid phase support. Test sera, mixed with a labeled reagent, is then reacted with the bound reference reagent. The reagents are then subjected to a series of dilution, incubation, and washing steps in order to separate bound and free reagents. The process concludes with a detection step, compatible with the type of label used, designed to indirectly measure the amount of antibody (or antigen) in the test sera.

“Mass spectrometry” refers to an analytical technique that measures the mass-to-charge ratio of ions to determine the masses of particles and of molecules in a sample. Mass spectrometry, whatever its type, generally includes steps consisting in identifying molecules present in a sample by measuring the mass of these molecules after they have been ionized, accelerated and introduced in a mass spectrometer.

“High-performance liquid chromatography” (HPLC) refers to a technique used in analytical chemistry to separate, identify, and quantify each component in a mixture. Typically, a pressurized liquid solvent containing the sample mixture is passed through a column filled with a solid adsorbent material. Each component in the sample interacts slightly differently with the adsorbent material, causing different flow rates for the different components and leading to the separation of the components as they flow out of the column.

“Lateral Flow Immunoassay”, also known as lateral flow immunochromatographic assays, Lateral flow test or Lateral flow device, refers to devices intended to detect the presence of a target analyte in liquid sample by running said liquid along the surface of a pad comprising a series of capillary beds, such as pieces of porous paper, microstructured polymer, or sintered polymer with reactive molecules that show a visual positive or negative result.

### SUMMARY OF THE INVENTION

The invention relates to a method for predicting the return to functional autonomy in a subject suffering from an acute event comprising the following steps:
i) determining neopterin level in a biological sample obtained from said subject;
ii) comparing said level with a predetermined reference neopterin level and
iii) predicting that the subject will have a long time to return to functional autonomy when the level of neopterin is higher than its predetermined reference neopterin level or predicting that the subject will have a short time to return to functional autonomy when the level of neopterin is lower than its predetermined reference neopterin level.

This method allows a physician to determine whether the subject will have a long time to return to functional autonomy and is therefore a subject in need of a specific care adapted to its situation, or a short time to return to functional autonomy and is therefore a subject which will recover with a standard level of care.

Consequently, a subject identified as having a predicted long time to return to functional autonomy will be monitored closely, its care regimen adapted to suit its need, or will be addressed to a specialized care unit, improving its recovery and its quality of life after the discharge from the hospital.

As used herein, the expression “short time to return to functional autonomy” indicates that the subject will have a time to return to functional autonomy that will be lower than the median (or mean) observed in the general population of subjects suffering from said acute event.

Inversely, the expression “long time to return to functional autonomy” indicates that the subject will have a time to return to functional autonomy that will be higher than the median (or mean) observed in the general population of subjects suffering from said disease.

In an embodiment of the invention, a short time to return to functional autonomy is comprised between five days to thirty days, while a long time to return to functional autonomy is comprised between thirty days to a year. In another embodiment of the invention, a short time to return to functional autonomy is comprised between five days to three months, while a long time to return to functional autonomy is comprised between three months to a year.

In an embodiment of the invention, the neopterin level measured in the biological sample is the neopterin concentration. In this embodiment, the predetermined reference neopterin level is the predetermined reference neopterin concentration.

In an embodiment of the invention, the time to return to functional autonomy is dependent on the neopterin level measured in the biological sample obtained from said subject.

In a preferred embodiment, the subject of the method of the invention wherein is a human subject. More preferably, said subject is an elderly subject, such as a subject older than 65 years old, more preferably older than 75 years old.

In a preferred embodiment, the acute event is selected from the group comprising hip fracture, pelvic fracture, tibia fracture, fibula fracture, foot fracture, wrist fracture, hand fracture.

In a preferred embodiment, the biological sample is selected from the group comprising a blood sample, a serum sample, a plasma sample, a urine sample, a saliva sample, a cerebrospinal fluid sample and a feces sample.

In a preferred embodiment, the biological sample is fresh, fresh frozen or frozen.

In a preferred embodiment, said biological sample is obtained at a time chosen from the group comprising:
a) Following medical management
b) Between the acute event and a surgery operation aimed to treat the acute event, preferably at hospital arrival,
c) During the surgery operation,
d) The day following the surgery operation,
e) From one to twelve days following the surgery operation, preferably from seven to ten days, and
f) From twelve days to twelve months following the surgery operation, preferably from six to twelve months.

In a preferred embodiment, said biological sample is obtained at hospital arrival.

Methods for determining a neopterin level in a biological sample are well known in the art. Examples of such methods include, but are not limited to, immunohistochemistry, multiplex methods (Luminex), enzyme-linked immunosorbent assay (ELISA), sandwich ELISA, fluorescent-linked immunosorbent assay (FLISA), enzyme immunoassay (EIA), radioimmunoassay (RIA), mass spectrometry (MS), a microarray, and the like, high-performance liquid chromatography or Lateral Flow Immunoassay, or any combination thereof.

In a preferred embodiment, step i) the method of the invention is performed by ELISA, mass spectrometry, high-performance liquid chromatography or Lateral Flow Immunoassay.

In a particular embodiment the neopterin level is measured by competitive enzyme-linked immunosorbent assay (ELISA) by following commercial kits (e.g. DRG Diagnostics, Biomnis) using horseradish peroxidase (HRPO)-labeled neopterin. Particularly, ELISA is suitable for measuring neopterin level in saliva, urine and blood sample.

In another embodiment the neopterin level is assayed by high pressure liquid chromatography (HPLC; see, e.g., Huber et al., J. Chromatography B: Biomed. Sci. App. 666(2): 223-232 (April 1995) regarding HPLC of neopterin in serum) with fluorescence detection after appropriate sample clean-up. Particularly, HPLC is performed to measure neopterin level in urine or plasma samples.

In one embodiment, the conclusion as to whether the subject will have a long or a short time to return to functional autonomy is taken after comparing the neopterin level of a specific sample of a subject following an acute event, preferably a bodily fluid sample, more preferably a blood sample to a predetermined reference value.

Typically, a reference neopterin level may be either implemented in a software or an overall median or other arithmetic mean across measurements may be built.

In one embodiment, the predetermined reference neopterin level is derived from the measurement of the neopterin levels according to the invention, in a control sample derived from a reference population.

In one embodiment, the reference population includes without limitation, such subjects having similar age range, the same sex, subjects in the same or similar ethnic group, and the like.

In one embodiment, the reference population comprises subjects, preferably at least 50, more preferably at least 100, more preferably at least 200 and even more preferably at least 500 subjects.

In one embodiment, the reference population comprises substantially healthy subject(s).

As used herein, a “substantially healthy subject” has not previously suffered from an acute event. In a preferred embodiment, a substantially healthy subject has not suffered from an acute event during the three months before collection of said control sample.

In a preferred embodiment, a substantially healthy subject is not treated with an immune system altering treatment.

In a preferred embodiment, a substantially healthy subject has not been the subject of partial or total arthroplasty.

In one embodiment, the reference population comprises subjects suffering from an acute event.

In a preferred embodiment, the reference population comprises subjects suffering from the same acute event as the subject to be treated.

In a preferred embodiment, the reference population does not include any person suffering from osteoporosis, or from an osteoporosis related acute event.

In one embodiment, the reference population comprises both subjects suffering from an acute event, more preferably of the same acute event as the subject to be treated, and substantially healthy subjects.

In an embodiment, the person skilled in the art define a predetermined reference neopterin level with a given method of measurement. In this embodiment, the predetermined reference neopterin level is the neopterin level obtained from a control sample or a reference population. In this embodiment, the neopterin level is determined with the same method of measurement as the predetermined reference neopterin level. Therefore, the person skilled in the art knows how to measure the neopterin level in a subject using a given method of measurement and to compare said neopterin level with a predetermined reference neopterin level obtained from a control sample or from a reference population to predict that the subject will have a long or short time to return to functional autonomy depending on the level of neopterin measured.

In another embodiment, the method of prediction comprises a step iii′ predicting that the subject will have a lower maximal recuperation score when the level of neopterin is higher than its predetermined reference neopterin level or predicting that the subject will have a greater maximal recuperation score when the level of neopterin is lower than its predetermined reference neopterin level.

In another embodiment, the method of prediction comprises a step iii′ predicting that the subject will have a longer time necessary to walk 5 m when the level of neopterin is higher than its predetermined reference neopterin level or predicting that the subject will have a shorter time necessary to walk 5 m when the level of neopterin is lower than its predetermined reference neopterin level.

In another embodiment, the method of prediction comprises a step iii′″ predicting that the subject will have a lower score obtained for the Short Physical Performance Battery when the level of neopterin is higher than its predetermined reference neopterin level or predicting that the subject will have a greater score obtained for the Short Physical Performance Battery when the level of neopterin is lower than its predetermined reference neopterin level.

In another embodiment, the method of prediction comprises a step iii′″ predicting that the subject will have a lower score obtained for the Activities of Daily Life when the level of neopterin is higher than its predetermined reference neopterin level or predicting that the subject will have a greater score obtained for the Activities of Daily Life when the level of neopterin is lower than its predetermined reference neopterin level.

In another embodiment, the method of prediction comprises a step iii′″ predicting that the subject will have a lower score obtained for the Instrumentalized Activities of Daily Life when the level of neopterin is higher than its predetermined reference neopterin level or predicting that the subject will have a greater score obtained for the Instrumentalized Activities of Daily Life when the level of neopterin is lower than its predetermined reference neopterin level.

In another embodiment, the predetermined reference neopterin level is superior to 10 nmol/L. Preferably, the predetermined reference neopterin level is comprised between 10 and 17 nmol/L, preferably 12 and 15 nmol/L. More preferably, the predetermined reference neopterin level is 15 nmol/L.

The invention also relates to a method of treatment, of a subject suffering from an acute event comprising the following steps:
i. determining neopterin level in a biological sample obtained from said subject;
ii. comparing said level with a predetermined reference neopterine level and
iii. predicting that the subject will have a long time to return to functional autonomy when the level of neopterin is higher than its predetermined reference neopterin level or predicting concluding that the subject will have a short time to return to functional autonomy when the level of neopterin is lower than its predetermined reference neopterin level.
iv. adapting the treatment of the subject according to the prediction of step iii.

In a preferred embodiment, step iv) comprises directing the subject to a specialized care unit. Additionally or alternatively, step iv) comprises adapting the regimen of physical exercises practiced by the patient, whether alone or under the action or supervision of a physician.

The invention also relates to a kit for predicting the return to functional autonomy in a subject suffering from an acute event.

In an embodiment of the invention, the kit comprises a collection mean for the sample, as well as the reagents necessary to carry out the method according to the first aspect of the invention.

The invention also relates to a Lateral Flow Immunoassay (LFI) device. The device comprises a sample pad—i.e. an area comprising a series of capillary beds, such as pieces of porous paper, microstructured polymer, or sintered polymer—and a conjugate pad in which the manufacturer has stored bio-active particles called conjugates in a matrix. The conjugate pad contains all the reagents required for an optimized chemical reaction between the target molecule (e.g., an antigen) and its chemical partner (e.g., antibody) that has been immobilized on the particle's surface. This marks target particles as they pass through the pad and continue across to the test and control lines. The test line shows a signal, often a color as in pregnancy tests. The test may also be quantitative and indicate a concentration of the target molecule.

Other advantages and features of the present invention will become readily apparent from the following detailed description of the invention.

### DESCRIPTION OF THE FIGURES

FIG. 1A-1C are a combination of graphs showing Neopterin levels in elderly at different times. The concentration of neopterin (in nmol/1) was measured at three different times (A) At hospital discharge; (B) at rehab discharge; (C) at six months post-fracture and analyzed for the hip fracture patients according to 2 clinical outcomes: able to walk (black squares, n=84; 28 and 24; respectively) or unable to walk (n=14; 18 and 59; respectively). p are calculated with a non-parametric Mann-Whitney test.

FIG. 2A-2C are a combination of graphs showing that Neopterin levels in elderly are linked to a time to return to functional autonomy. The concentration of neopterin of the patients was measured at arrival to hospital (in nmol/1), and compared to three clinical parameters at different time (A) Maximal recovery at hospital discharge (B) Walking Delay at 30 days after arrival at hospital (C) Short Physical Performance Battery (SPPB) at 30 days after arrival at hospital. p are calculated with a non-parametric Mann-Whitney test.

FIG. 3A-3C are a combination of graphs showing that Neopterin levels in elderly are linked to a time to return to functional autonomy, with others tests and indexes with reference to FIGS. 2A-2C. The concentration of neopterin of the patients was measured at arrival to hospital (in nmol/1), and compared to three clinical parameters at different time (A) Activities of Daily Life (B) Instrumentalized Activities of Daily Life (C) Short Physical Performance Battery (SPPB) at 30 days after surgery. p are calculated with a non-parametric Mann-Whitney test.

FIG. 4 is a Receiver Operating Characteristics (ROC) curve depicting sensitivity and specificity of prediction model for Functional Autonomy-related to ability to walk at D30 based on neopterin concentration measured at arrival to hospital. AUC=0.91; Specificity=100%; Sensitivity=75%; threshold: =15 nM.

### DETAILED DESCRIPTION—EXAMPLE

More than two hundred elderly individuals >75 years old were screened to participate in our study. One third of the cohort was constituted of healthy individuals whereas the other ones were admitted in emergency department for hip fracture. Of note, patients suffering from osteoporosis were not included; therefore only accidental hip fracture patients were included in our cohort (n=120). Longitudinal follow-up post fracture was designed with blood samples taken at their arrival to hospital (DO; PRE), after surgery (POST), at their discharge from the hospital (D7-D10; EXIT) and at long-term post fracture (M6-M12; FOLLOW UP).

Plasma as well as PBMCs have been cryopreserved until use. Experiments always included samples from healthy controls compared to hip-fracture patients (all timepoints being performed simultaneously).

We choose to focus on the level of neopterin reached by the patients at their arrival to hospital.

We analyzed neopterin as a predictive marker of functional recovery post hip fracture, according to the following protocol.

Materials and Methods

Experiments were performed using a commercially available kit from IBL-international by following manufacturer's instructions (described below).

Principle of the Test

Neopterin ELISA kit is based on competitive binding of human Neopterin from serum samples and enzyme-labeled Neopterin to Neopterin specific antibodies immobilized on microtiter plates. After a washing step, chromogenic substrate is added and color developed. The enzymatic reaction (blue color) is inversely proportional to the amount of Neopterin present in the sample. The reaction is terminated by adding stopping solution (converts blue to yellow). Absorbance is then measured on an ELISA reader at 450 nm and the concentration of Neopterin in samples and control is read off the standard curve.

Sensitivity

The lower detection limit is calculated from the standard curve by determining the resulting concentration of the mean OD of Calibrator A (based on 10 replicate analyses) minus 2 SD. The sensitivity of the Neopterin ELISA kit is 0.7 nmol/L.

Specificity (Cross Reactivity)

The following compounds were tested for cross-reactivity with the Direct Neopterin ELISA kit. No significant interference was detected at the following concentration. Hemoglobin 35 mg/dL, Bilirubin 2.25 mg/d£, Triglyceride 125 mg/dL.

Specimen Collection and Handling

Blood was collected by venipuncture, allowed to clot, and serum was separated by centrifugation at room temperature. The serum was not heat inactivate. If sera cannot be immediately assayed, these could be stored at −20° C. for up to six months. While the sample can be frozen and thawed, repeated freezing and thawing of samples should be avoided. Do not use specimens containing NaN3. Samples appearing turbid should be centrifuged before testing to remove any particulate material.

Our experiments were performed on frozen human plasma. The plasma samples have been aliquoted, protected from light and cryopreserved at −80° C. for one year before usage. Thawing has been done by putting samples in a fridge allowing slow thawing. Thereafter, thawed plasma have been centrifuged at 1000 rpm during 10 min in the dark.

Reagents Preparation

Stock Wash buffer was diluted (1:20) with water and stored at 4° C. for 1 month.

All reagents were at room temperature prior to their use.

For the following experiment, human plasma aliquots were used undiluted and manipulated in the dark. If necessary, samples to be used would have been diluted with assay buffer (1:10).

Storage and Stability

The microtiter well plate and all other reagents are stable at 2-8° C. until the expiration date printed on the label. The whole kit stability is usually 6 months from the date of shipping under appropriate storage conditions. The unused portions of the standards should be stored at 2-8° C. or stored frozen in small aliquots.

Test Procedure

All reagents were allowed to reach room temperature before use. The required number of coated strips were removed and arranged on the microtiter well plate.

The microtiter well strips to be used on the plate were labeled and the wash buffer was diluted with water (1:20).

20 μl of standards, controls, and samples were pipeted into appropriate wells in duplicate.

100 μl of ready-to-use enzyme conjugate were added into each well, followed by 50 μl of ready-to-use Neopterin antiserum into each well, before gently mixing for 5-10 seconds. The plate was covered and incubated for 90 minutes at 18-25° C. on orbital shaker (500 rpm) in the dark.

The well contents were aspirated and the plate blotted on absorbent paper before immediately washing the wells 4 times with 300 μl of 1× wash buffer.

150 μl of TMB substrate solution was added before gently mixing for 5-10 seconds. The plate was covered and incubated for 10 mins in the dark at 18-25° C.

The reaction was stopped by adding 150 μl of stop solution to all wells at the same timed intervals as in step 5. Gentle mixing was performed for 5-10 seconds to have uniform color distribution (blue color turned yellow).

Absorbance was measured at 450 nm using an ELISA reader within 15 min.

Calculation of Results

The absorbance of all duplicates were averaged before subtracting the averaged non-specific binding (NSB) absorbance from the average obtained above. This yields the net absorbance. Net absorbance was divided by the net zero standard absorbance (Bo) to obtain the percent bound (% B/Bo).

Formula:

Abs. (sample)−Abs. (NSB)

% B/Bo=×100 Abs. (zero standard)−Abs. (NSB)

Abs.=average absorbance of duplicate wells

NSB=non-specific binding (also known as the blank) Sample=particular serum or standard being calculated Zero Standard=0 nmol/L standard or 100% binding wells.

Construct a plot of the percent bound (Y-axis) versus the concentration of the neopterin standards ((X-axis) starting with the 0.5 nmol/L point. Either logit-log or semi-log graph paper may be used. This yields the standard curve.

Using the standard curve, the neopterin concentration of each sample was determined.

Expected Reference Neopterin Level

Usually, healthy subjects show the following values: healthy subjects neopterin level (Normal): <10 nmol/L (0.3-3.0 ng/mL).

Conversion: Neopterin (nmol/L)×0.253=ng/mL

Results & Discussion

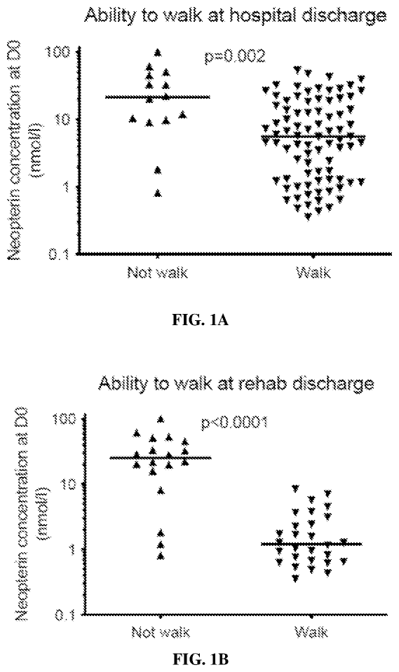

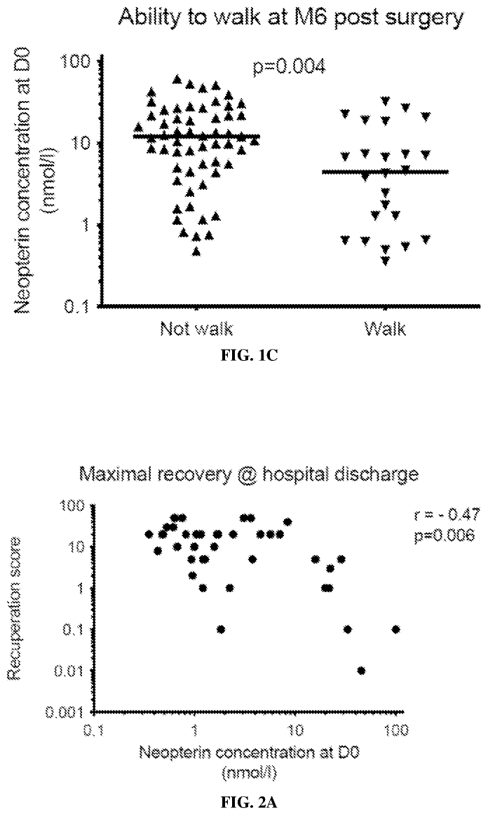

As shown in FIG. 1, the level of neopterin was statistically different between the hip fracture patients who were able to walk compared to those who were unable to perform the tests. This observation was valid for their ability to walk at hospital discharge (FIG. 1A.; p=0.002), after their stay in rehab unit (FIG. 1B.; p<0.0001), and at Month 6 post fracture (FIG. 1C.; p=0.004).

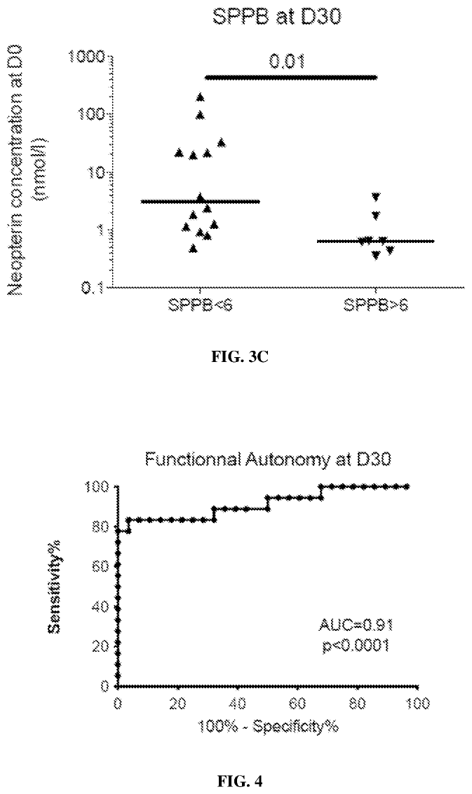

A cutoff value between a population of functionally autonomous subjects and a population of dependent subjects was determined at 15 nmol/L (75% sensitivity; 100% specificity; AUC=0.91) based on Receiver Operating Characteristic analysis related to ability to walk at D30 post surgery (FIG. 4).

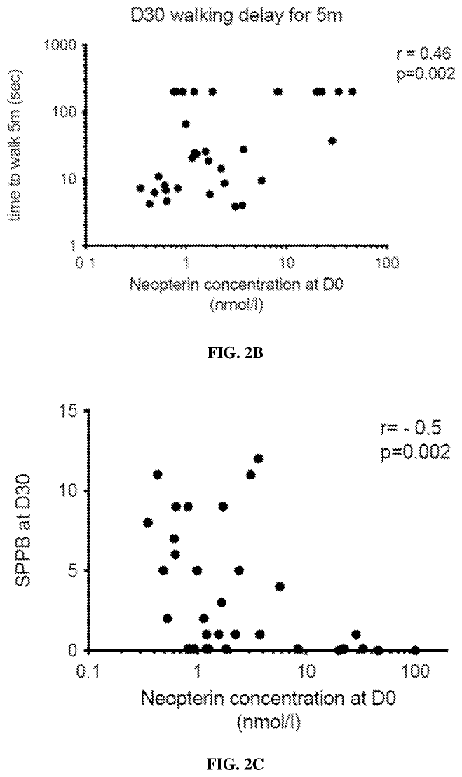

We also found correlations between the level of neopterin measured at arrival to hospital and:
the maximal recuperation score (FIG. 2A, p=0.006; r=−0.47)
the time necessary to walk 5 m at day 30 post-surgery (FIG. 2B, p=0.002; r=0.46)
the score obtained for the Short Physical Performance Battery (SPPB) measured at D30 post-surgery (FIG. 2C, p=0.002; r=−0.5)

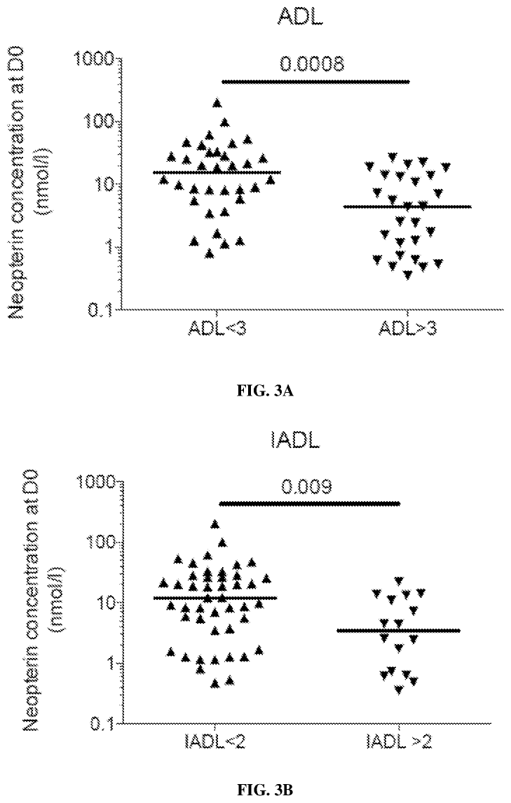

Additional comparisons between the level of neopterin measured at arrival to hospital and functional autonomy of the subjects were performed, emphasizing the correlation between neopterin and return to functional autonomy:
the score obtained for the Activities of Daily Life (ADL) measured at D30 post-surgery (FIG. 3A, p=0.0008)
the score obtained for the Instrumentalized Activities of Daily Life (IADL) measured at D30 post-surgery (FIG. 3B, p=0.009)
the score obtained for the Short Physical Performance Battery (SPPB) measured at D30 post-surgery (FIG. 3C, p=0.01)

1-15. (canceled)

16. A method for predicting the return to functional autonomy in a subject suffering from an acute event comprising the following steps:
i) determining neopterin level in a biological sample obtained from said subject;
ii) comparing said level with a predetermined reference neopterin level; and
iii) predicting that the subject will have a long time to return to functional autonomy when the level of neopterin is higher than its predetermined reference neopterin level or predicting that the subject will have a short time to return to functional autonomy when the level of neopterin is lower than its predetermined reference neopterin level.

17. The method according to claim 16, wherein the subject is a human subject.

18. The method according to claim 17, wherein the subject is an elderly subject.

19. The method according to claim 18, wherein the subject is older than 65 years old.

20. The method according to claim 17, wherein the subject is older than 75 years old.

21. The method according to claim 16, wherein the acute event is selected from the group comprising hip fracture, pelvic fracture, tibia fracture, fibula fracture, foot fracture, wrist fracture, clavicle fracture and hand fracture.

22. The method according to claim 21, wherein the acute event is hip fracture.

23. The method according to claim 16, wherein the biological sample is selected from the group comprising a blood sample, a serum sample, a plasma sample, an urine sample, a saliva sample, a cerebrospinal fluid sample and a feces sample.

24. The method according to claim 23, wherein the biological sample is a blood sample.

25. The method according to claim 16, wherein the biological sample is fresh, fresh frozen or frozen.

26. The method according to claim 16, wherein the biological sample is obtained at a time chosen from the group comprising:
(a) following medical management,
(b) between the acute event and a surgery operation aimed to treat the acute event,
(c) during the surgery operation,
(d) the day following the surgery operation,
(e) from one to twelve days following the surgery operation, and
(f) from twelve days to twelve months following the surgery operation.

27. The method according to claim 26, wherein the biological sample is obtained at hospital arrival.

28. The method according to claim 26, wherein the biological sample is obtained from seven to ten days following the surgery operation.

29. The method according to claim 26, wherein the biological sample is obtained from six to twelve months following the surgery operation.

30. The method according to claim 16, wherein step i) is performed by ELISA, mass spectrometry, high-performance liquid chromatography or Lateral Flow Immunoassay.

31. The method according to claim 16, wherein the predetermined reference neopterin level is superior to 10 nmol/L.

32. The method according to claim 31, wherein the predetermined reference neopterin level is comprised between 10 and 17 nmol/L.

33. The method according to claim 31, wherein the predetermined reference neopterin level is comprised between 12 and 15 nmol/L.

34. The method according to claim 31, wherein the predetermined reference neopterin level is 15 nmol/L.

35. A kit for predicting the return to functional autonomy in a subject suffering from an acute event.

36. The kit according to claim 35, comprising:
a collection mean for the sample, and
the reagents necessary to carry out a method for predicting the return to functional autonomy in a subject suffering from an acute event comprising the following steps:
i) determining neopterin level in a biological sample obtained from said subject;
ii) comparing said level with a predetermined reference neopterin level; and
iii) predicting that the subject will have a long time to return to functional autonomy when the level of neopterin is higher than its predetermined reference neopterin level or predicting that the subject will have a short time to return to functional autonomy when the level of neopterin is lower than its predetermined reference neopterin level.
